(19) United States
(12) Patent Application Publication
(10) Pub. No.: US 2007/0259281 A1
(43) Pub. Date: Nov. 8, 2007

(54) Hydroxygallium phthalocyanines

(75) Inventors: Liang-Bih Lin, Rochester, NY (US); Francisco J. Lopez, Rochester, NY (US); Daniel V. Levy, Rochester, NY (US); Jin Wu, Webster, NY (US); Yushan Kim, Troy, MI (US); John S. Chambers, Rochester, NY (US)

Correspondence Address: George Likourezos, Esq.;Carter, DeLuca, Farrell & Schmidt, LLP, Suite 225, 445 Broad Hollow Rd., Melville, NY, 11747, US

(73) Assignee: Xerox Corporation

(21) Appl. No.: 11/429,500

(22) Filed: May 5, 2006

(51) Int. Cl. G03G 5/06; C07D 487/22

(52) U.S. Cl. 430/58.8; 430/78; 430/59.4; 540/140

(57) ABSTRACT

Methods for preparing Type V hydroxygallium phthalocyanine are provided.

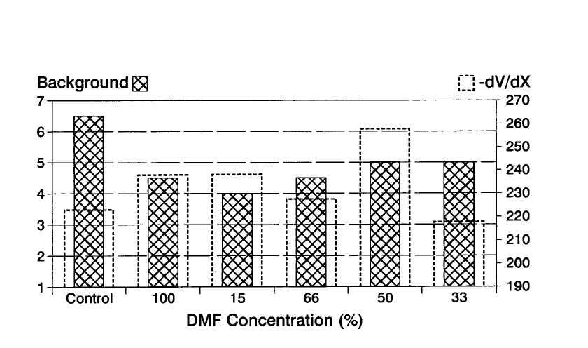

### BACKGROUND

The disclosure relates to phthalocyanines and more specifically to phthalocyanine dyes, colorants, like pigments, and mixtures thereof for use in photoreceptors and, more particularly, to methods for the production of hydroxygallium phthalocyanines.

Hydroxygallium phthalocyanine (HOGaPc) pigments are currently utilized in photoreceptors. HOGaPc polymorphs are known, including the Type V polymorph, also known as Type V hydroxygallium phthalocyanine or Type V HOGaPc. U.S. Pat. Nos. 5,521,306 and 5,473,064, the disclosures of each of which are hereby incorporated by reference in their entirety, describe HOGaPc and processes to prepare Type V HOGaPc. Type V HOGaPc has been characterized by its intense diffraction peaks at Bragg angles 7.5, 9.9, 12.5, 16.3, 18.6, 21.9, 23.9, 25.1, and 28.3, with the highest peak at 7.5 degrees 2Θ (2 theta±0.2°) in the X-ray diffraction spectrum. HOGaPc is most responsive at a range of, for example, from about 550 nanometers to about 880 nanometers and is generally unresponsive to the light spectrum below about 500 nanometers. Typical wavelengths for photogeneration may be from about 600 nanometers to about 850 nanometers and may include a broadband between the two wavelengths. Single wavelength exposure may be from about 750 nanometers to about 850 nanometers.

There are certain drawbacks that may be associated with the photoreceptor use of HOGaPc, including high dark decay characteristics and a tendency of inducing charge deficient spots (CDS). Large HOGaPc particles of a size larger than about 500 nm in the charge generation layer may be a cause of these problems, which results in poor image quality.

Therefore, HOGaPc pigments made of smaller particles of a size from about 5 nm to about 450 nm, and thus possessing a larger surface area and which also possess high sensitivity, are desirable. It is believed that raw pigments that for example, possess a higher surface area from about 5 m²/g to about 120 m²/g would result in charge generation layers with pigments having finer particle sizes, as the conversion of raw pigments, such as Type I polymorph of HOGaPc to the Type V polymorph, only changes surface properties of the crystal and not crystal size.

### SUMMARY

The present disclosure provides processes which, in embodiments, include contacting a gallium phthalocyanine in an acid solution with a basic aqueous media to form a slurry, contacting the slurry with a washing agent selected from the group consisting of ketones, ethers, alkanes, glycols, alcohols, aromatics, and pyridines to form a pigment, and contacting the resulting pigment with a solvent system including at least two solvents of polar aprotic solvents, esters, and ketones.

In embodiments, the process includes contacting a gallium phthalocyanine with an acid solution at a molar concentration of from about 5 molar to about 30 molar and a basic aqueous media having a molar concentration from about 3 molar to about 15 molar to form a slurry, contacting the slurry with a suitable washing agent, and contacting the resulting pigment with a solvent system including at least two polar aprotic solvents, esters, and ketones.

In still other embodiments, the process includes contacting an alkoxy-bridged gallium phthalocyanine in an acid solution which may be hydrogen halides, oxyacids of halogens and/or organic sulfonic acids, with ammonia to form a pigment slurry, concentrating the pigment slurry by filtration to obtain a pigment filtrate, contacting the pigment filtrate with a washing agent which may be ketones, ethers, alkanes, glycols, alcohols, aromatics, and pyridines to obtain a pigment, contacting the resulting pigment with a solvent system including at least two solvents selected from at least two groups including a polar aprotic solvent such as N,N-dimethylformamide, N-methylpyrrolidone, dimethyl sulfoxide, acetonitrile, and mixtures thereof, an ester which may be n-butyl acetate, ethyl acetate, and mixtures thereof, and a ketone which may be acetone, methyl ethyl ketone, methyl isobutyl ketone, and mixtures thereof.

Photoreceptors possessing pigments produced by methods herein are also provided. In embodiments, a photoreceptor may include a photogenerating layer including a resin and a photogenerating component including a hydroxygallium phthalocyanine prepared by contacting a gallium phthalocyanine in an acid solution with a basic aqueous media to form a pigment slurry, contacting the pigment slurry with a washing agent such as ketones, ethers, alkanes, glycols, alcohols, aromatics, and pyridines to form a pigment, and contacting the resulting pigment with at least two solvents selected from at least two of the groups including polar aprotic solvents, esters, and ketones.

### BRIEF DESCRIPTION OF THE DRAWINGS

Various embodiments of the present disclosure will be described herein below with reference to the FIGURE wherein:

The FIGURE is a graph depicting results from electrical scanning and print testing of photoreceptors produced with Type V HOGaPc polymorphs prepared in accordance with the present disclosure with varying concentrations of dimethylformamide (DMF) and methyl ethyl ketone (MEK) utilized in converting Type I HOGaPc to Type V HOGaPc.

### EMBODIMENTS

The present disclosure provides processes for the preparation of hydroxygallium phthalocyanine, especially the Type V polymorph. The methods of the present disclosure produce an amorphous Type I HOGaPc which may then be converted to high surface area Type V HOGaPc. High surface area Type V HOGaPc refers, in embodiments, for example, to HOGaPc having a surface area of from about 5 m²/g to about 120 m²/g, in embodiments from about 30 m²/g to about 80 m²/g. Methods for measuring surface area are known and include, for example, the Brunauer, Emmett and Teller (BET) method or mercury porosimetry. The methods of the present disclosure result in a reduction in the particle size and/or agglomerate size of Type I HOGaPc, which aids in achieving the desired smaller particle size and particle size distribution of the resulting Type V HOGaPc. Because of the smaller particle size, there may be a reduction in the production milling time by about 50%, in embodiments from about 10% to about 50%, in embodiments from about 20% to about 40%, and excellent print quality may be achieved when such phthalocyanines are incorporated as photogenerating layers in layered imaging members.

Utilizing the methods of the present disclosure, one can improve charge deficient spot performance of hydroxygallium phthalocyanine by, for example, introducing an additional washing of a Type I polymorph of hydroxygallium phthalocyanine pigment prior to its final conversion to Type V, a very photosensitive form of the pigment.

The methods of the present disclosure may be utilized to convert a Type I HOGaPc obtained by any method to a Type V HOGaPc. In embodiments, the Type I HOGaPc may be obtained by a process disclosed in U.S. Pat. No. 5,473,064, the disclosure of which is hereby incorporated by reference in its entirety. Such a process includes, in embodiments, a process for the preparation of hydroxygallium phthalocyanine, essentially free of a halide like chlorine, whereby a pigment precursor Type I halogallium phthalocyanine, in embodiments a chlorogallium phthalocyanine, is prepared by the reaction of gallium chloride in a solvent, such as N-methylpyrrolidone, present in an amount of from about 10 parts to about 100 parts, in embodiments from about 15 to about 25 parts, and in embodiments about 19 parts, with 1,3-diiminoisoindoline in an amount of from about 1 part to about 10 parts, in embodiments about 4 parts, of 1,3-diiminoisoindoline, for each part of gallium chloride that is reacted; hydrolyzing the pigment precursor chlorogallium phthalocyanine Type I by standard methods, for example acid pasting, whereby the pigment precursor is dissolved in an acid such as sulfuric acid and then reprecipitated in a solvent, such as water, or a dilute ammonia solution, for example from about 10 to about 15 percent, to obtain the resulting hydrolyzed pigment hydroxygallium phthalocyanine Type I.

In embodiments, the process of the present disclosure also includes the conversion of Type I hydroxygallium phthalocyanine to Type V hydroxygallium phthalocyanine wherein the Type I hydroxygallium phthalocyanine is prepared by the hydrolysis of a dimer. This process includes the dissolution of 1 part gallium chloride in from about 1 part to about 100 parts, in embodiments from about 5 parts to about 15 parts, of an organic solvent. Suitable organic solvents include, for example, aromatics including benzene, toluene, xylene and the like. The reaction can occur at a temperature of from about 0° C. to about 100° C., in embodiments at a temperature of from about 20° C. to about 30° C., to form a solution of gallium chloride. The gallium chloride solution may be contacted with from about 1 part to about 5 parts, in embodiments from about 2 parts to about 4 parts, of an alkali metal alkoxide such as sodium methoxide, sodium ethoxide, sodium propoxide or the like, in embodiments in a solution form, to produce a gallium alkoxide solution and an alkali metal salt byproduct, for example sodium chloride. The reaction can occur at a temperature of from about 0° C. to about 100° C., in embodiments at a temperature of from about 20° C. to about 40° C.

The alkali metal salt byproduct may be removed from the resulting gallium alkoxide solution by reaction with from about 1 part to about 10 parts, in embodiments from about 2 parts to about 6 parts, orthophthalodinitrile or 1,3-diiminoisoindolene, and a diol, such as 1,2-ethanediol (ethylene glycol), 1,2-propanediol (propylene glycol) or 1,3-propanediol, in an amount of from about 3 parts to about 100 parts, in embodiments from about 5 parts to about 15 parts, for each part of gallium alkoxide formed. The reaction can occur at a temperature of from about 150° C. to about 220° C., in embodiments at a temperature of from about 185° C. to about 205° C., for a period of from about 30 minutes to about 6 hours, in embodiments from about 1 hour to about 3 hours, to provide an alkoxy-bridged gallium phthalocyanine dimer pigment precursor. This dimer pigment may be isolated by filtration at a temperature of from about 20° C. to about 180° C., in embodiments from about 100° C. to about 140° C.

The dimer precursor may then be added to a concentrated acid, such as sulfuric acid, hydrogen halides including hydrochloric acid (HCl), hydrobromic acid (HBr), hydroiodic acid (HI), oxyacids of halogens including chloric acid (HClO3), perchloric acid (HClO4), bromic acid (HBrO3), perbromic acid (HBrO4), iodic acid (HIO3), periodic acid (HIO4), nitric acid, and/or trifluoroacetic acid, to form a solution. The acid may be at a molar concentration from about 5 molar to about 30 molar, in embodiments from about 10 molar to about 20 molar. The acid may be at any suitable pH. In embodiments, the acid may have a pH lower than about 1. In embodiments, an acid such as an organic sulfonic acid may be used. Suitable organic sulfonic acids include, but are not limited to, methanesulfonic acid, ethanesulfonic acid, propanesulfonic acid, pentanesulfonic acid, hexanesulfonic acid, heptanesulfonic acid, pyridinesulfonic acid, chloroethanesulfonic acid, bromoethanesulfonic acid, 1-diazo-2-naphthol-4-sulfonic acid, 3-hydroxypropane-1-sulfonic acid, aniline sulfonic acid, and combinations thereof. In embodiments, a more solvable acid such as an organic sulfonic acid may be utilized to produce the desired Type I hydroxygallium phthalocyanine.

The use of such an organic sulfonic acid may produce a high quality, low CDS pigment capable of maintaining excellent photosensitivity as well as low discharged surface potential at an exposure range of about 2 to about 4 ergs/cm².

Halogenated organic solvents may be added to dissolve the dimer, wherein the volume/volume ratio of the acid to the halogenated solvent is from about 1/10 to about 10/1, in embodiments from about 1/1 to about 5/1. Examples of halogenated solvents include alkylkene halides, like methylene chloride, chloroform, 1,2-dichloroethane, 1,1,2-tricloroethane, and monochlorobenzene. The alkoxy-bridged gallium phthalocyanine dimer pigment can be dissolved in the concentrated acid in an amount of from about 1 weight part to about 100 weight parts, in embodiments from about 25 weight parts to about 75 weight parts, by stirring said pigment in the acid for an effective period of time, from about one minute to about 24 hours, in embodiments from about 2 hours to about 4 hours. The temperature of the solution can be from about 0° C. to about 75° C., in embodiments from about 40° C. to about 60° C., in air or under an inert atmosphere such as argon or nitrogen. The resulting pigment slurry may be filtered through a 5 μm glass filter to remove any insoluble pigments.

The resulting pigment slurry may be added at a controlled rate to a solvent and reprecipitated in a basic aqueous media in what may be referred to, in embodiments, as an acid pasting step. The solvent may include aqueous solvents, such as aqueous hydroxides for example ammonia, aqueous sodium hydroxide, and the like. These solvents may be utilized in a wash to reprecipitate and provide an alkoxy-bridged gallium phthalocyanine dimer. Each different diol used for the phthalocyanine synthesis will produce a particular alkoxy-bridged gallium phthalocyanine dimer product, as determined by, for example, infrared (IR) spectroscopy, nuclear magnetic resonance (NMR) and X-ray powder diffraction pattern (XRD). In embodiments, where sulfuric acid is utilized to dissolve the alkoxy-bridged gallium phthalocyanine, the resulting dissolved pigment may be reprecipitated in aqueous ammonia to form a pigment slurry.

The basic solution may be of from about 3 molar to about 15 molar concentration, in embodiments of from about 6 molar to about 10 molar concentration, selecting from about 1 volume part to about 10 volume parts of the basic solution for each volume part of acid that was used. The basic solution may be at any suitable pH. In embodiments, the basic solution may possess a pH greater than about 13.

The solvents may be chilled while being stirred during pigment precipitation in order to maintain a temperature of from about −20° C. to about 40° C., in embodiments from about 0° C. to about 10° C., during pigment precipitation. The resulting pigment may be isolated by, for example, filtration. In embodiments, the filtrate may be washed with deionized water to obtain a filtrate of a neutral pH.

The resulting hydroxygallium phthalocyanine possesses in embodiments, for example, X-ray diffraction patterns having major peaks at Bragg angles (2 theta±0.2°) of 6.8, 7.0, 13.5, 16.6, 23.8, 26.7, and 28.1 referred to, in embodiments, as Type I hydroxygallium phthalocyanine.

The Type I hydroxygallium phthalocyanine pigment may then be subjected to washing in accordance with the present disclosure. Suitable washing agents which may be utilized in this washing include, but are not limited to, ketones including alkyl alkyl ketones such as methyl ethyl ketone, ethers including dialkyl ethers such as diethyl ether, alkanes including those having from about 10 to about 40 carbon atoms such as dodecane, glycols including alkylene glycols such as ethylene glycol and propylene glycol, alcohols including those having from about 2 carbon atoms to about 20 carbon atoms such as ethanol and propanol, aromatics including substituted aromatics having from about 1 to about 4 functional groups such as methyl, amino, carboxy, nitro, and the like, examples of which include toluene and xylene, and pyridines including alkyl pyridines such as 2-methylpyridine and 2-ethyl pyridine, and combinations thereof. In some embodiments, the washing agent utilized for this washing step includes a methyl ethyl ketone. After this washing, the Type I hydroxygallium phthalocyanine may be separated from the washing agent utilizing any method within the purview of one skilled in the art including, but not limited to, filtration, centrifugation, and the like. In embodiments, the Type I hydroxygallium phthalocyanine may then be dried prior to any further treatment by, for example, vacuum drying at a temperature of from about 60° C. to about 100° C., in embodiments from about 70° C. to about 90° C., for a period of time from about 30 minutes to about 5 hours, in embodiments from about 1 hour to about 3 hours.

Without wishing to be bound by any theory, it is believed this additional washing in combination with optional sonocrystallization, may impart a texture to the surface of the Type I pigment. This “roughing” up of the Type I pigment results in excellent and homogeneous conversion of the Type I pigment to the Type V pigment. In accordance with the present disclosure, it is believed that the added washing after hydrolysis but prior to conversion results in a more amorphous Type I HOGaPc. For example, Type I HOGaPc pigments produced in accordance with the present disclosure that are subjected to this washing demonstrate an absorption peak of from about 4% to about 6% at about 680 nm when subjected to UV-visible absorption spectroscopy, indicating an amorphous pigment.

After washing, the Type I hydroxygallium phthalocyanine can be contacted or treated with a mixed solvent system to convert Type I HOGaPc to Type V HOGaPc. The combined solvents of the solvent system include an excellent conversion solvent with a poor one, which allows for a more uniform crystal particle size and structure. The size of the resulting particles may be from about 5 nm to about 450 nm, in embodiments from about 10 nm to about 200 nm, in other embodiments from about 50 nm to about 150 nm. The ratio between the excellent conversion and poor conversion solvents can be adjusted for a proper conversion rate so that uniform particle size and crystal structure can be obtained. The slower conversion rate allows for the pigment particles to break down to the smallest size possible prior to complete conversion. This reduces the presence of agglomerates which have centers of unconverted Type I HOGaPc.

In embodiments, the solvent system may include at least two of a polar aprotic solvent, an ester and/or a ketone. Suitable polar aprotic solvents include N,N-dimethylformamide (DMF), N-methylpyrrolidone, dimethyl sulfoxide, acetonitrile, mixtures thereof, and the like. Suitable esters include n-butyl acetate, ethyl acetate, mixtures thereof, and the like. Suitable ketones include acetone, methyl ethyl ketone (MEK), methyl isobutyl ketone, mixtures thereof, and the like.

The polar aprotic solvent may be present in the solvent system in an amount from about 1 percent to about 99 percent by weight of the solvent system, in embodiments from about 20 percent to about 80 percent by weight of the solvent system. The ester may be present in the solvent system in an amount from about 1 percent to about 99 percent by weight of the solvent system, in embodiments from about 20 percent to about 50 percent by weight of the solvent system. The ketone may be present in the solvent system in an amount from about 5 percent to about 80 percent by weight of the solvent system, in embodiments from about 10 percent to about 70 percent by weight of the solvent system. In embodiments, the components of the solvent system, for example the polar aprotic solvent, ester and/or ketone, add up to about 100 percent by weight of the solvent system. In embodiments, the solvent system may include a polar aprotic solvent and ester, a polar aprotic solvent and ketone, an ester and ketone, or a polar aprotic solvent, ester, and ketone.

At least two of the solvents are independently selected from the above three classes to form the solvent system of the present disclosure, in embodiments from about 2 to about 7 solvents may be utilized, in embodiments from about 3 to about 5 solvents may be utilized, as long as one solvent is from a different class of solvents than the other solvent(s).

The Type I HOGaPc may be contacted with the solvent system of the present disclosure, optionally by, for example, stirring, ball milling or otherwise contacting said Type I hydroxygallium phthalocyanine pigment with the aforementioned solvent system in the absence or presence of grinding media such as stainless steel shot, spherical or cylindrical ceramic media, or spherical glass beads. The Type I HOGaPc product may be combined with the solvent system and grinding media at a temperature of from about 0° C. to about 40° C., in embodiments from about 10° C. to about 30° C., for a period of from about 2 hours to about 2 weeks, in embodiments from about 72 hours to about 1 week, with constant rolling speed from about 30 rpm to about 150 rpm, in embodiments from about 50 rpm to about 70 rpm.

In embodiments, the Type I HOGaPc may be placed in the solvent system and combined with glass beads for milling. Suitable glass beads include, for example, from about 1 mm to about 6 mm soda lime glass beads (Glen Mills, Inc., Clifton, N.J.), and borosilicate glass beads (HiBea D20 from Ohara Inc., Kanagawa, Japan). From about 2 grams to about 100 grams of Type I HOGaPc, in embodiments from about 5 grams to about 7 grams of the Type I HOGaPc, may be contacted with from about 16 grams to about 800 grams of a solvent system such as DMF and MEK, in embodiments from about 40 grams to about 80 grams of the solvent system, with from about 60 grams to about 3000 grams of beads, in embodiments from about 160 grams to about 200 grams of beads, and milled from about 2 hours to about 2 weeks, in embodiments from about 72 hours to about 1 week, with constant rolling speeds from about 30 rpm to about 150 rpm, in embodiments from about 50 rpm to about 70 rpm.

The resulting high surface area Type V HOGaPc may be separated from the mixture by washing with DMF, acetone, or mixtures thereof followed by filtration. The Type V polymorph may then be dried under vacuum at a temperature of from about 50° C. to about 95° C., in embodiments from about 65° C. to about 80° C. and then crushed utilizing ball milling, rotary milling, and the like.

The resulting Type V HOGaPc possesses an X-ray diffraction pattern having major peaks at Bragg angles (2 theta±0.2°) of 7.2, 10, 16.8, 18.6, 24, 25.3, 26.8, 28.3, 32.5 and with the highest peak at 7.2 degrees 2Θ.

The surface area of the resulting Type V hydroxygallium phthalocyanine can be from about 5 m²/g to about 120 m²/g, in embodiments from about 30 m²/g to about 80 m²/g. The high surface area of the Type V hydroxygallium phthalocyanine of the present disclosure may contribute to pigment dispersibility when the pigment is combined with a resin to form a photogenerating layer of a photoreceptor.

Methods which may be utilized to determine the surface area of the HOGaPc include, for example, the Brunauer, Emmett and Teller (BET) method or mercury porosimetry. In embodiments, a multi point BET method using nitrogen as the adsorbate may be utilized.

In the BET process, about 0.5 grams of a sample may be weighed into analysis vessels. The samples may be degassed at about 50° C. under full vacuum overnight, in embodiments from about 12 to about 20 hours, prior to analysis. The surface area may be determined using nitrogen as the adsorbate gas at 77 Kelvin (LN2), over a relative pressure range of from about 0.08 to about 0.25 using a Micromeritics ASAP 2405 surface area instrument. The surface area is the BET surface area minus the micropore area. Micropores are pores with a diameter of 20 angstroms or less. Micropore areas may be calculated for the HOGaPc pigments using the Harkins and Jura method over the specified thickness range of 6 to 10 angstroms. The surface area obtained by this method is comparable to the surface area as determined by mercury porosimetry.

In embodiments, sonocrystallization may be utilized to achieve the desired pigment size of the resulting Type V HOGaPc. Sonocrystallization includes, for example, the use of ultrasound technology to induce nucleation in crystal formation, which may result in the production of crystals having a smaller size. In embodiments, the sonocrystallization may be conducted prior to the conversion of the Type I pigment to the Type V pigment. In other embodiments the sonocrystallization may be conducted after the conversion of the Type I pigment to the Type V pigment. In embodiments, sonocrystallization may also result in the production of crystals having a narrow particle size distribution of from about 5 nm to about 1000 nm, in embodiments from about 10 nm to about 200 nm.

For example, in embodiments the Type I hydroxygallium phthalocyanine may be mixed with N-dimethylformamide and, after agitated in a roller for a period of time from about 20 minutes to about 120 minutes, in embodiments from about 30 minutes to about 90 minutes, methyl ethyl ketone may be added. Subsequently, power ultrasound of from about 0.5 to about 10 MHz, in embodiments of from about 1 to about 5 MHz, may be applied to the Type I hydroxygallium phthalocyanine in combination with the solvent system of the present disclosure, and then glass beads may be added and the container rolled at from about 30 to about 150 revolutions per minute, in embodiments from about 50 to about 120 revolutions per minute, for a time from about 30 hours to about 200 hours, in embodiments from about 80 hours to about 160 hours. The resultant slurry may be collected by separating the beads and slurry and washing same with N-dimethylformamide followed by methyl ethyl ketone in a suction filter. The resulting pigment may then be vacuum dried at from about 50° C. to about 100° C. for a time from about 6 to about 48 hours.

Sonocrystallization may allow the crystals to grow in a more controlled way, which provides a narrower particle size distribution, as indicated by the milling time and the absorption spectra obtained for a dispersion possessing such a pigment.

The hydroxygallium phthalocyanine pigments produced in accordance with the present disclosure may be combined with a resin to form a charge generation layer of a photoreceptor. Examples of suitable resins for use in preparing the dispersion include thermoplastic and thermosetting resins such as polycarbonates, polyesters including poly(ethylene terephthalate), polyurethanes including poly(tetramethylene hexamethylene diurethane), polystyrenes including poly(styrene-co-maleic anhydride), polybutadienes including polybutadiene-graft-poly(methyl acrylate-co-acrylontrile), polysulfones including poly(1,4-cyclohexane sulfone), polyarylethers including poly(phenylene oxide), polyarylsulfones including poly(phenylene sulfone), polyethersulfones including poly(phenylene oxide-co-phenylene sulfone), polyethylenes including poly(ethylene-co-acrylic acid), polypropylenes, polymethylpentenes, polyphenylene sulfides, polyvinyl acetates, polyvinylbutyrals, polysiloxanes including poly(dimethylsiloxane), polyacrylates including poly(ethyl acrylate), polyvinyl acetals, polyamides including poly(hexamethylene adipamide), polyimides including poly(pyromellitimide), amino resins including poly(vinyl amine), phenylene oxide resins including poly(2,6-dimethyl-1,4-phenylene oxide), terephthalic acid resins, phenoxy resins including poly(hydroxyethers), epoxy resins including poly([(o-cresyl glycidyl ether)-co-formaldehyde], phenolic resins including poly(4-tert-butylphenol-co-formaldehyde), polystyrene and acrylonitrile copolymers, polyvinylchlorides, polyvinyl alcohols, poly-N-vinylpyrrolidinones, vinylchloride and vinyl acetate copolymers, carboxyl-modified vinyl chloride/vinyl acetate copolymers, hydroxyl-modified vinyl chloride/vinyl acetate copolymers, carboxyl- and hydroxyl-modified vinyl chloride/vinyl acetate copolymers, acrylate copolymers, alkyd resins, cellulosic film formers, poly(amideimide), styrene-butadiene copolymers, vinylidenechloride-vinylchloride copolymers, vinylacetate-vinylidenechloride copolymers, styrene-alkyd resins, polyvinylcarbazoles, and the like, and mixtures thereof. These polymers may be block, random, or alternating copolymers.

Examples of suitable polycarbonates which may be utilized to form the charge generation layer dispersion include, but are not limited to, poly(4,4′-isopropylidene diphenyl carbonate) (also referred to as bisphenol A polycarbonate), poly(4,4′-diphenyl-1,1′-cyclohexane carbonate) (also referred to as bisphenol Z polycarbonate, polycarbonate Z, or PCZ), poly(4,4′-sulfonyl diphenyl carbonate) (also referred to as bisphenol S polycarbonate), poly(4,4′-ethylidene diphenyl carbonate) (also referred to as bisphenol E polycarbonate), poly(4,4′-methylidene diphenyl carbonate) (also referred to as bisphenol F polycarbonate), poly(4,4′-(1,3-phenylenediisopropylidene)diphenyl carbonate) (also referred to as bisphenol M polycarbonate), poly(4,4′-(1,4-phenylenediisopropylidene)diphenyl carbonate) (also referred to as bisphenol P polycarbonate), poly(4,4′-hexafluoroisppropylidene diphenyl carbonate).

Examples of suitable vinyl chlorides and vinyl acetates which may be utilized to form the dispersion utilized to form the charge generation layer include, but are not limited to, carboxyl-modified vinyl chloride/vinyl acetate copolymers such as VMCH (available from Dow Chemical) and hydroxyl-modified vinyl chloride/vinyl acetate copolymers such as VAGF (available from Dow Chemical).

The weight molecular weight of the resin used to form the charge generation layer may be for example, from about 10,000 to about 100,000, in embodiments from about 15,000 to about 50,000.

In embodiments, a single resin may be utilized to form the charge generation layer. In other embodiments, a mixture of more than one of the above resins can be used to form the charge generation layer. Where more than one resin is utilized, the number of resins can be from about 2 to about 4, in embodiments from about 2 to about 3.

A liquid or liquid mixture may be used in preparing the charge generation layer. A liquid mixture may include from about 2 to about 4 liquids, in embodiments from about 2 to about 3 liquids. In embodiments, the liquid is a solvent for the resin, but not the high surface area Type V HOGaPc of the present disclosure. The resin may be added to the liquid, in embodiments a solvent for the resin, to form a solution and the pigment then added to the solution to form a dispersion suitable for forming the charge generation layer. The liquid utilized should not substantially disturb or adversely affect other layers of the photoreceptor, if any. Examples of liquids that can be utilized in preparing the charge generation layer include, but are not limited to, ketones, alcohols, aromatic hydrocarbons, halogenated aliphatic hydrocarbons, ethers, amines, amides, esters, mixtures thereof, and the like. Specific illustrative examples include cyclohexanone, acetone, methyl ethyl ketone, methanol, ethanol, butanol, amyl alcohol, toluene, xylene, monochlorobenzene, carbon tetrachloride, chloroform, methylene chloride, trichloroethylene, tetrahydrofuran, dioxane, diethyl ether, dimethyl formamide, dimethyl acetamide, butyl acetate, ethyl acetate, methoxyethyl acetate, mixtures thereof, and the like.

The resin in a liquid, which is a solvent for the resin, is combined with the hydroxygallium, such as Type V HOGaPc prepared in accordance with the present disclosure. Any suitable technique may be utilized to disperse the Type V HOGaPc in the resin or resins. The dispersion containing the pigment may be formed using, for example, attritors, ball mills, Dynomills, paint shakers, homogenizers, microfluidizers, mechanical stirrers, in-line mixers, ultrasonic processor, Cavipro processor, or by any other suitable milling techniques.

Specific dispersion techniques which may be utilized include, for example, ball milling, roll milling, milling in vertical or horizontal attritors, sand milling, and the like. The solids content of the mixture being milled can be selected from a wide range of concentrations. Milling times using a ball roll mill may be from about 6 hours to about 6 days, in embodiments from about 8 hours to about 3 days. However, as noted above, in embodiments milling of large particles is not required as the methods of the present disclosure result in Type V HOGaPc having a high surface area.

The amount of resin in the dispersion can be from about 95% by weight to about 15% by weight of the solids, in embodiments from about 65% by weight to about 20% by weight of the solids. The amount of pigment in the dispersion can be from about 5% by weight to about 85% by weight of the dispersion solids, in embodiments from about 35% by weight to about 80% by weight of the dispersion solids. The expression “solids” refers to the total pigment and resin components of the dispersion.

Any suitable and conventional technique may be utilized to apply the dispersion of the present disclosure to form a charge generation layer on another layer of a photoreceptor. Suitable coating techniques include dip coating, roll coating, spray coating, rotary atomizers, and the like.

The charge generation layer containing the pigments of the present disclosure and the resinous material may be of a thickness from about 0.05 μm to about 5 μm, in embodiments from about 0.1 μm to about 1 μm, although the thickness can be outside these ranges. The charge generation layer thickness is related to the relative amounts of pigment and resin, with the pigment often being present in amounts from about 5 to about 80 percent by weight, in embodiments from about 45 to about 70 percent by weight. Higher resin content compositions generally require thicker layers for photogeneration. Generally, it may be desirable to provide this layer in a thickness sufficient to absorb about 90 percent or more of the incident radiation which is directed upon it in the imagewise or printing exposure step. The maximum thickness of this layer depends upon factors such as mechanical considerations, the thicknesses of the other layers, and whether a flexible photoconductive imaging member is desired.

The dispersions of the present disclosure may be utilized to form charge generation layers in conjunction with any known configuration for photoreceptors, including single and multi-layer photoreceptors. Examples of multi-layer photoreceptors include those described in U.S. Pat. Nos. 6,800,411, 6,824,940, 6,818,366, 6,790,573, and U.S. Patent Application Publication No. 20040115546, the disclosures of each of which are hereby incorporated by reference in their entirety. Photoreceptors may possess a charge generation layer (CGL), also referred to herein as a photogenerating layer, and a charge transport layer (CTL). Other layers, including a substrate, an electrically conductive layer, a charge blocking or hole blocking layer, an adhesive layer, and/or an overcoat layer, may also be present in the photoreceptor.

Suitable substrates which may be utilized in forming a photoreceptor include opaque or substantially transparent substrates, and may include any suitable organic or inorganic material having the requisite mechanical properties.

The substrate may be flexible, seamless, or rigid and may be of a number of different configurations such as, for example, a plate, a cylindrical drum, a scroll, an endless flexible belt, a web, and the like.

The thickness of the substrate layer may depend on numerous factors, including mechanical performance and economic considerations. For rigid substrates, the thickness of the substrate can be from about 0.3 millimeters to about 10 millimeters, in embodiments from about 0.5 millimeters to about 5 millimeters. For flexible substrates, the substrate thickness can be from about 65 to about 200 micrometers, in embodiments from about 75 to about 100 micrometers, for optimum flexibility and minimum stretch when cycled around small diameter rollers of, for example, 19 millimeter diameter. The entire substrate can be made of an electrically conductive material, or the electrically conductive material can be a coating on a polymeric substrate.

Substrate layers selected for the imaging members of the present disclosure, and which substrates can be opaque or substantially transparent, may include a layer of insulating material including inorganic or organic polymeric materials such as MYLAR® (a commercially available polymer from DuPont), MYLAR® containing titanium, a layer of an organic or inorganic material having a semiconductive surface layer, such as indium tin oxide or aluminum arranged thereon, or a conductive material inclusive of aluminum, chromium, nickel, brass, or the like.

Any suitable electrically conductive material can be employed with the substrate. Suitable electrically conductive materials include copper, brass, nickel, zinc, chromium, stainless steel, conductive plastics and rubbers, aluminum, semi-transparent aluminum, steel, cadmium, silver, gold, zirconium, niobium, tantalum, vanadium, hafnium, titanium, nickel, chromium, tungsten, molybdenum, paper rendered conductive by the inclusion of a suitable material therein, or through conditioning in a humid atmosphere to ensure the presence of sufficient water content to render the material conductive, indium, tin, metal oxides, including tin oxide and indium tin oxide, and the like.

After formation of an electrically conductive surface, a hole blocking layer may optionally be applied to the substrate layer. Generally, hole blocking layers (also referred to as charge blocking layers) allow electrons from the conductive layer to migrate toward the charge generation layer. Any suitable blocking layer capable of forming an electronic barrier to holes between the adjacent charge generation layer and the underlying conductive layer of the substrate may be utilized. Suitable blocking layers include those disclosed, for example, in U.S. Pat. Nos. 4,286,033, 4,291,110 and 4,338,387, the disclosures of each of which are hereby incorporated by reference in their entirety. Similarly, illustrated in U.S. Pat. Nos. 6,255,027, 6,177,219, and 6,156,468, the disclosures of each of which are hereby incorporated by reference in their entirety, are, for example, photoreceptors containing a hole blocking layer of a plurality of light scattering particles dispersed in a binder resin. For example, Example 1 of U.S. Pat. No. 6,156,468 discloses a hole blocking layer of titanium dioxide dispersed in a linear phenolic binder.

Hole blocking layers utilized for negatively charged photoreceptors may include, for example, polyamides including LUCKAMIDE® (a nylon type material derived from methoxymethyl-substituted polyamide commercially available from Dai Nippon Ink), hydroxy alkyl methacrylates, nylons, gelatin, hydroxyl alkyl cellulose, organopolyphosphazines, organosilanes, organotitanates, organozirconates, metal oxides of titanium, chromium, zinc, tin, silicon, and the like. In embodiments the hole blocking layer may include nitrogen containing siloxanes. Nitrogen containing siloxanes may be prepared from coating solutions containing a hydrolyzed silane. Hydrolyzable silanes include 3-aminopropyl triethoxy silane, N,N′-dimethyl 3-amino) propyl triethoxysilane, N,N-dimethylamino phenyl triethoxy silane, N-phenyl aminopropyl trimethoxy silane, trimethoxy silylpropyldiethylene triamine and mixtures thereof.

In embodiments, the hole blocking components may be combined with phenolic compounds, a phenolic resin, or a mixture of more than one phenolic resin, for example, from about 2 to about 4 phenolic resins. Suitable phenolic compounds which may be utilized may contain at least two phenol groups, such as bisphenol A (4,4′-isopropylidenediphenol), bisphenol E (4,4′-ethylidenebisphenol), bisphenol F (bis(4-hydroxyphenyl)methane), bisphenol M (4,4′-(1,3-phenylenediisopropylidene)bisphenol), bisphenol P (4,4′-(1,4-phenylene diisopropylidene)bisphenol), bisphenol S (4,4′-sulfonyldiphenol), and bisphenol Z (4,4′-cyclohexylidenebisphenol), hexafluorobisphenol A (4,4′-(hexafluoro isopropylidene)diphenol), resorcinol, hydroxyquinone, catechin, and the like.

The hole blocking layer may be applied as a coating on a substrate or electrically conductive layer by any suitable conventional technique such as spraying, die coating, dip coating, draw bar coating, gravure coating, silk screening, air knife coating, reverse roll coating, vacuum deposition, chemical treatment, and the like. For convenience in obtaining thin layers, the blocking layers may be applied in the form of a dilute solution, with the solvent being removed after deposition of the coating by conventional techniques such as by vacuum, heating and the like. Drying of the deposited coating may be effected by any suitable conventional technique such as oven drying, infrared radiation drying, air drying and the like.

The blocking layer may be continuous and have a thickness of from about 0.01 micrometers to about 30 micrometers, in embodiments from about 0.1 micrometers to about 20 micrometers.

An optional adhesive layer may be applied to the hole blocking layer. Any suitable adhesive layer known in the art may be utilized including, but not limited to, polyesters, polyamides, poly(vinyl butyral), poly(vinyl alcohol), polyurethane and polyacrylonitrile. Where present, the adhesive layer may be, for example, of a thickness of from about 0.001 micrometers to about 2 micrometers, in embodiments from about 0.01 micrometers to about 1 micrometer. Optionally, the adhesive layer may contain effective suitable amounts, for example from about 1 weight percent to about 10 weight percent, of conductive and nonconductive particles, such as zinc oxide, titanium dioxide, silicon nitride, carbon black, and the like, to provide further desirable electrical and optical properties to the photoreceptor of the present disclosure. Conventional techniques for applying an adhesive layer coating mixture to the hole blocking layer include spraying, dip coating, roll coating, wire wound rod coating, gravure coating, die coating and the like. Drying of the deposited coating may be effected by any suitable conventional technique such as oven drying, infrared radiation drying, air drying and the like.

In embodiments the photoreceptor also includes a charge transport layer attached to the charge generation layer. The charge transport layer may include a charge transport or hole transport molecule (HTM) dispersed in an inactive polymeric material. These compounds may be added to polymeric materials which are otherwise incapable of supporting the injection of photogenerated holes from the charge generation layer and incapable of allowing the transport of these holes therethrough. The addition of these HTMs converts the electrically inactive polymeric material to a material capable of supporting the direction of photogenerated holes from the charge generation layer and capable of allowing the transport of these holes through the charge transport layer in order to discharge the surface charge on the charge transport layer.

Suitable polymers for use in forming the charge transport layer are known film forming resins. Examples include those polymers utilized to form the charge generation layer. In embodiments resin materials for use in forming the charge transport layer are electrically inactive resins including polycarbonate resins having a weight average molecular weight from about 20,000 to about 150,000, in embodiments from about 50,000 about 120,000. Electrically inactive resin materials which may be utilized in the charge transport layer include poly(4,4′-dipropylidene-diphenylene carbonate) with a weight average molecular weight of from about 35,000 to about 40,000, available as LEXAN® 145 from General Electric Company; poly(4,4′-propylidene-diphenylene carbonate) with a weight average molecular weight of from about 40,000 to about 45,000, available as LEXAN® 141 from the General Electric Company; a polycarbonate resin having a weight average molecular weight of from about 50,000 to about 100,000, available as MAKROLON® from Farbenfabricken Bayer A.G.; a polycarbonate resin having a weight average molecular weight of from about 20,000 to about 50,000 available as MERLON® from Mobay Chemical Company; and a polycarbonate resin having a weight average molecular weight of from about 20,000 to about 80,000 available as PCZ from Mitsubishi Chemicals. Solvents such as methylene chloride, tetrahydrofuran, toluene, monochlorobenzene, or mixtures thereof, may be utilized in forming the charge transport layer coating mixture.

Any suitable charge transporting or electrically active molecules may be employed as HTMs in forming a charge transport layer on a photoreceptor. Suitable charge transporting molecules include, for example, aryl amines as disclosed in U.S. Pat. No. 4,265,990, the disclosure of which is hereby incorporated by reference in its entirety. In embodiments, an aryl amine charge hole transporting component may be represented by: 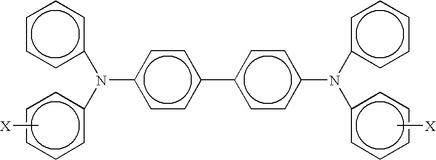
wherein X can be alkyl, halogen, alkoxy or mixtures thereof. In embodiments, the halogen is a chloride. Alkyl groups may contain, for example, from about 1 to about 10 carbon atoms and, in embodiments, from about 1 to about 5 carbon atoms. Examples of suitable aryl amines include, but are not limited to, N,N′-diphenyl-N,N′-bis(alkylphenyl)-1,1-biphenyl-4,4′-diamine, wherein the alkyl may be methyl, ethyl, propyl, butyl, hexyl, and the like; and N,N′-diphenyl-N,N′-bis(halophenyl)-1,1′-biphenyl-4,4′-diamine, wherein the halo may be a chloro, bromo, fluoro, and the like substituent.

Other suitable aryl amines which may be utilized as an HTM in a charge transport layer include, but are not limited to, tritolylamine, N,N′-bis(3,4 dimethylphenyl)-N″(1-biphenyl)amine, 2-bis((4′-methylphenyl)amino-p-phenyl) 1,1-diphenyl ethylene, 1-bisphenyl-diphenylamino-1-propene, triphenylmethane, bis(4-diethylamine-2-methylphenyl)phenylmethane, 4′-4″-bis(diethylamino)-2′,2″-dimethyltriphenylmethane, N,N′-bis(alkylphenyl)-[1,1′-biphenyl]-4,4′-diamine wherein the alkyl is, for example, methyl, ethyl, propyl, n-butyl, etc., N,N′-diphenyl-N,N′-bis(3″-methylphenyl)-(1,1′-biphenyl)-4,4′-diamine, and the like.

The weight ratio of the polymer resin to charge transport molecules in the resulting charge transport layer can be, for example, from about 30/70 to about 80/20. In embodiments the weight ratio of the polymer resin to charge transport molecules can be from about 35/65 to about 75/25, in embodiments from about 40/60 to about 70/30.

Any suitable and conventional technique may be utilized to mix the polymer resin in combination with the hole transport material and apply same as a charge transport layer to a photoreceptor. In embodiments, it may be advantageous to add the polymer resin and hole transport material to a solvent to aid in formation of a charge transport layer and its application to a photoreceptor. Examples of solvents which may be utilized include aromatic hydrocarbons, aliphatic hydrocarbons, halogenated hydrocarbons, ethers, amides and the like, or mixtures thereof. In embodiments, a solvent such as cyclohexanone, cyclohexane, chlorobenzene, carbon tetrachloride, chloroform, methylene chloride, trichloroethylene, toluene, tetrahydrofuran, dioxane, dimethyl formamide, dimethyl acetamide and the like, may be utilized in various amounts. Application techniques of the charge transport layer include spraying, slot or slide coating, dip coating, roll coating, wire wound rod coating, and the like. Drying of the deposited coating may be effected by any suitable conventional technique such as oven drying, infrared radiation drying, air drying and the like.

The thickness of the charge transport layer can be from about 2 micrometers and about 50 micrometers, in embodiments from about 10 micrometers to about 35 micrometers. The charge transport layer should be an insulator to the extent that the electrostatic charge placed on the charge transport layer is not conducted in the absence of illumination at a rate sufficient to prevent formation and retention of an electrostatic latent image thereon. In general, the ratio of the thickness of the charge transport layer to the charge generation layer, where present, is in embodiments from about 2:1 to 200:1 and in some instances as great as 400:1.

The charge generation layer, charge transport layer, and other layers may be applied in any suitable order to produce either positive or negative charging photoreceptors. For example, the charge generation layer may be applied prior to the charge transport layer, as illustrated in U.S. Pat. No. 4,265,990, or the charge transport layer may be applied prior to the charge generation layer, as illustrated in U.S. Pat. No. 4,346,158, the disclosures of each of which are hereby incorporated by reference in their entirety. When used in combination with a charge transport layer, the charge generation layer may be sandwiched between a conductive surface and a charge transport layer or the charge transport layer may be sandwiched between a conductive surface and a charge generation layer.

Optionally, an overcoat layer may be applied to the surface of a photoreceptor to improve resistance to abrasion. In some cases, an anti-curl back coating may be applied to the side of the substrate opposite the active layers of the photoreceptor (i.e., the CGL and CTL) to provide flatness and/or abrasion resistance where a web configuration photoreceptor is fabricated. These overcoating and anti-curl back coating layers are known and may include thermoplastic organic polymers or inorganic polymers that are electrically insulating or slightly semi-conductive. For example, overcoat layers may be fabricated from a dispersion including a particulate additive in a resin. Suitable particulate additives for overcoat layers include metal oxides including aluminum oxide, non-metal oxides including silica or low surface energy polytetrafluoroethylene, and mixtures thereof. Suitable resins include those described above as suitable for charge generation layers and/or charge transport layers, for example, polyvinyl acetates, polyvinylbutyrals, polyvinylchlorides, vinylchloride and vinyl acetate copolymers, carboxyl-modified vinyl chloride/vinyl acetate copolymers, hydroxyl-modified vinyl chloride/vinyl acetate copolymers, carboxyl- and hydroxyl-modified vinyl chloride/vinyl acetate copolymers, polyvinyl alcohols, polycarbonates, polyesters, polyurethanes, polystyrenes, polybutadienes, polysulfones, polyarylethers, polyarylsulfones, polyethersulfones, polyethylenes, polypropylenes, polymethylpentenes, polyphenylene sulfides, polysiloxanes, polyacrylates, polyvinyl acetals, polyamides, polyimides, amino resins, phenylene oxide resins, terephthalic acid resins, phenoxy resins, epoxy resins, phenolic resins, polystyrene and acrylonitrile copolymers, poly-N-vinylpyrrolidinones, acrylate copolymers, alkyd resins, cellulosic film formers, poly(amideimide), styrene-butadiene copolymers, vinylidenechloride-vinylchloride copolymers, vinylacetate-vinylidenechloride copolymers, styrene-alkyd resins, polyvinylcarbazoles, and mixtures thereof. Overcoatings may be continuous and have a thickness from about 0.5 micrometers to about 10 micrometers, in embodiments from about 2 micrometers to about 6 micrometers.

An example of an anti-curl backing layer is described in U.S. Pat. No. 4,654,284, the disclosure of which is hereby incorporated by reference in its entirety. In embodiments, it may be desirable to coat the back of the substrate with an anticurl layer such as, for example, polycarbonate materials commercially available as MAKROLON® from Bayer Material Science. The thickness of anti-curl backing layers should be sufficient to substantially balance the total forces of the layer or layers on the opposite side of the supporting substrate layer. A thickness for an anti-curl backing layer from about 10 micrometers to about 100 micrometers, in embodiments from about 15 micrometers to about 50 micrometers, is a satisfactory range for flexible photoreceptors.

The Type V hydroxygallium phthalocyanine obtained according to the present disclosure exhibits excellent properties in photoresponsive imaging members when used as a pigment, in particular, lower print background, lower charge deficient spots (CDS), and lower dark decay and better cyclic stability compared to lower surface area Type V hydroxygallium phthalocyanine obtained via previously utilized processes, for example, from dimer or other gallium phthalocyanine precursors such as, for example, chlorogallium phthalocyanine.

A reduction in the particle size and/or agglomerate size of the Type V HOGaPc precursor, Type I HOGaPc, aided in achieving the desired smaller particle size and particle size distribution required for Type V HOGaPc to reduce production milling time and most importantly improvement in print quality when used for the charge generation layer.

Processes of imaging, especially xerographic imaging and printing, are also encompassed by the present disclosure. More specifically, photoreceptors of the present disclosure can be selected for a number of different known imaging and printing processes including, for example, electrophotographic imaging processes, especially xerographic imaging and printing processes wherein charged latent images are rendered visible with toner compositions of an appropriate charge polarity. In embodiments, the imaging members may be sensitive in the wavelength region of, for example, from about 500 to about 900 nanometers, typically from about 650 to about 850 nanometers; thus diode lasers can be selected as the light source. Moreover, the imaging members of this disclosure may be useful in color xerographic applications, particularly high-speed color copying and printing processes.

The following Examples are being submitted to illustrate embodiments of the present disclosure. These Examples are intended to be illustrative only and are not intended to limit the scope of the present disclosure. Also, parts and percentages are by weight unless otherwise indicated.

### EXAMPLES

### Example 1

About 380 grams of methanesulfonic acid (CH₃SO₃H_{(conc.)}) was added in a 500 ml flask, and heated to about 50° C. About 7.7 grams of alkoxygallium phthalocyanine (ROGaPC) was then added to the solvent and stirred with a magnetic bar for about 2 hours. Care was taken to maintain the temperature from about 50° C. to about 60° C. The solution was then filtered using a Büchner Funnel having a pore size of about 4 to about 8 μm. Separately, about 430 grams of concentrated ammonia and about 170 grams of deionized water were added in a 2 L round glass container and placed into an acetone/dry ice bath.

The filtered acid solution was then gradually quenched by addition into the ammonia solution, and the pigment started to precipitate; care was taken to maintain the temperature below a temperature of from about 5° C. to about 10° C. Afterward, the slurry was suction filtered and the resulting pigment cake washed with deionized water until the conductivity of the wash water as measured by a conductivity meter (Orion 0115) was below about 30 μS.cm. The pigment cake was then collected and dried under vacuum at a temperature of about 80° C. overnight (from about 12 to about 20 hours), and about 7.3 grams of Type I HOGaPc was produced.

About 6 grams of the Type I HOGaPc pigment was then added into a 250 ml amber glass bottle with about 39 grams of dimethylformamide (DMF) and about 21 grams of acetone and about 250 grams of 1 mm diameter glass beads. The bottle was placed in a roller and rolled at a bottle speed of about 60 revolutions per minute (rpm) for about 5 days (about 120 hours). The resulting slurry was suction filtered and then washed twice with about 30 grams of DMF and washed twice with about 30 grams of acetone. The pigment cake was then dried under vacuum at a temperature of about 82° C. overnight (from about 12 to about 20 hours), and about 5.3 grams of Type V HOGaPc was generated.

The Type V HOGaPc pigment was bead milled in an attritor with a vinylacetate/vinyl chloride copolymer at a weight ratio of about 60:40 with a solids concentration of about 12% for about one hour. The mill base was than let down to about 5% with n-butyl acetate to produce a charge generating layer dispersion. The particle size of the dispersion was from about 50 nm to about 150 nm.

Devices were prepared as follows. An aluminum pipe having a diameter of about 30 mm was pre-coated with a silane-based undercoating layer having a thickness of about 1 μm. A charge transporting layer, including N,N′-diphenyl-N,N′-bis(alkylphenyl)-1,1-biphenyl-4,4′-diamine arylamine and poly(4,4′-diphenyl-1,1′-cyclohexane carbonate) (PCZ), having a thickness of from about 11 μm to about 13 μm was applied thereto. The charge generating layer dispersion described above was then applied by dip coating forming a charge generation layer having a thickness from about 0.1 to about 0.3 μm. For comparison, devices were prepared having the same configuration as above, but utilizing HOGaPc prepared from an alkoxygallium phthalocyanine (ROGaPc) precursor using a conventional conversion method of treatment of Type I HOGaPc with DMF alone to obtain Type V HOGaPc (denoted HOGaPc-RC) and HOGaPc prepared from a chlorogallium phthalocyanoine precursor using the same conventional conversion method of treatment of Type I HOGaPc with DMF alone to obtain Type V HOGaPc (denoted HOGaPc-CC), or utilizing laboratory scale pigments prepared wherein sulfuric acid (H₂SO₄) was used in the hydrolysis step instead of methanesulfonic acid. Thus, the devices tested were as follows: Device 1 was a drum having a 11 μm CTL and a CGL utilizing HOGaPc-RC; Device 2 was a drum having a 13 μm CTL and a CGL utilizing HOGaPc-RC pigment; Device 3 was a drum having a 11 μm CTL and a CGL prepared with a pigment obtained by hydrolyzing Type I HOGaPc with sulfuric acid; Device 4 was a drum having a 13 μm CTL and a CGL prepared with a pigment obtained by hydrolyzing Type I HOGaPc with sulfuric acid; Device 5 was a drum having a 11 μm CTL and a CGL prepared with a pigment obtained by hydrolyzing Type I HOGaPc with methanesulfonic acid; Device 6 was a drum having a 13 μm CTL and a CGL prepared with a pigment obtained by hydrolyzing Type I HOGaPc with methanesulfonic acid; Device 7 was a drum having a 11 μm CTL and a CGL utilizing HOGaPc-CC pigment; and Device 8 was a drum having a 13 μm CTL and a CGL utilizing HOGaPc-CC pigment.

The electrical properties of all these devices were obtained with a photoelectrical scanner and characterized as follows: dv/dx is the slope of a photoinduced discharge characteristic (PIDC) curve obtained for each device; V(2.8) was a V_{low} of 2.8 ergs/cm² exposure energy in volts; V(4.5) was a V_{low} of 4.5 ergs/cm² exposure energy in volts. Charge Deficiency Spot (CDS) performance was evaluated using a Xerox DC2240 copier situated in an environmental chamber conditioned at about 80% relative humidity (RH) and about 30° C. and printing at about 52 mm/sec process speed, which would be about the most severe case for generating CDS. CDS was measured by a visual scale rated from 1-7, where the higher the number, the worse the CDS. The electrical properties and CDS grades for all the experimental devices are set forth in Table 1 below.

TABLE 1
Electrical Properties and CDS Performance
of the experimental devices

| Device | dV/dX | V(2.8) | V(4.5) | CDS |
|---|---|---|---|---|
| Device 1 | 203 | 201 | 55 | 7++ |
| Device 2 | 240 | 139 | 25 | 7+ |
| Device 3 | 194 | 218 | 60 | 5 |
| Device 4 | 226 | 158 | 31 | 4 |
| Device 5 | 205 | 192 | 40 | 4.5 |
| Device 6 | 239 | 129 | 19 | 3 |
| Device 7 | 201 | 205 | 57 | 4 |
| Device 8 | 238 | 145 | 30 | 3 |

The electrical properties of all the devices exhibited nominal behaviors, but the pigment generated using methanesulfonic acid (CH₃SO₃H) showed lower V_{low} (evidenced by the V(2.8) and V(4.5) data in Table 1). As can be seen in Table 1, for the device utilizing the HoGaPc-CC pigment in the charge generation layer and the 13 μm charge transporting layer, V(2.8) and V(4.5) were about 145V±10 V and about 30 V±5 V, respectively. As a comparison, for the device utilizing the CH₃SO₃H generated pigment, the V(2.8) & V(4.5) were about 129 V and about 19 V, respectively, which were similar or even slightly better than the results obtained for the charge generation layer prepared with the HOGaPc-RC pigment.

The charge generating layer possessing pigment generated with CH₃SO₃H had only a 0.5 grade increase relative to the charge generating layer possessing the pigment from HOGaPc-CC, but with a much better (lower) V_{low}. In contrast, both the charge generating layers possessing pigment obtained from HOGaPc-RC and pigment obtained from laboratory scale H₂SO₄ hydrolysis had a much higher CDS grade, Grade 5 and Grade 7+, respectively.

The above results demonstrate, for example, a method of lowering CDS level without deteriorating photoelectrical performance, especially in photosensitivity and V_{low} by replacing the conventional sulfuric acid (H₂SO₄) with methanesulfonic acid for hydrolyzing ROGaPC pigment. The acid dissolved the pigment significantly better than H₂SO₄ and also provided excellent CDS performance when compared with the HOGaPc-CC pigment.

### Example 2

About 360 grams of concentrated sulfuric acid (H₂SO_{4(conc.)}) was added in a 500 ml flask, heat was applied to raise the temperature of the acid to about 50° C., then about 12 grams of ROGaPC was added to the solvent and stirring with a magnetic bar for about 2 hours. Care was taken to maintain the temperature of the solution from about 50° C. to about 60° C. The solution was then filtered using a Büchner Funnel having a pore size from about 4 to about 8 μm. Separately, about 800 grams of concentrated ammonia and about 300 grams of deionized water were added in a 2 L round glass container and put into an acetone/dry ice bath. The filtered acid solution was then gradually quenched into the ammonia solution, where pigment started to precipitate; care was taken to maintain the temperature below a temperature of from about 5° C. to about 10° C. Afterward, the slurry was suction filtered and the pigment cake washed with deionized water until the conductivity of the washed water was below about 30 μS.cm. The pigment cake was then collected and dried under vacuum at a temperature from about 80° C. to about 90° C. overnight (from about 12 to about 20 hours), and about 11.2 grams of Type I HOGaPc was produced.

The HOGaPc (I) was then subjected to a wash step of the present disclosure. In a 120 ml amber glass bottle, about 60 grams of methyl ethyl ketone was added with about 6 grams of the Type I HOGaPc obtained as described above and rolled in a roller for about 2 hours. The solvent was then filtered and the pigment was vacuum dried for about 2 hours at about 80° C.

The dried pigment was then added into a 250 ml amber glass bottle with about 39 grams of dimethylformamide (DMF) and about 21 grams of acetone and about 250 grams of about 1 mm diameter glass beads. The bottle was rolled in a roller at about 60 rpm bottle speed for about 5 days (120 hours), and the slurry was suction filtered and then washed twice with about 30 grams of DMF followed by washing twice with about 30 grams of acetone. The resulting pigment cake was then dried under vacuum at about 82° C. overnight (from about 12 to about 20 hours), and about 5.1 grams of Type V HOGaPc was generated.

The resulting Type V HOGaPc pigment was bead milled in an attritor with a vinylacetate/vinyl chloride copolymer at a weight ratio of about 60:40 with a solids content of about 12% for an hour. The mill base was then let down to about 5% using n-butyl acetate.

Devices were prepared by dip coating the charge generating layer dispersion on about a 30 mm diameter aluminum pipe pre-coated with a silane-based undercoating layer about 1 μm thick and then a charge transporting layer about 11 or 13 μm thick including an arylamine and polycarbonate. For comparison, devices were prepared having the same configuration, but utilizing laboratory scale DMF conversion of Type I HOGaPc pigments produced from alkoxygallium phthalocyanine (denoted HOGaPc-RC) and laboratory scale DMF conversion of Type I HOGaPc pigments produced from chlorogallium phthalocyanine (denoted HOGaPc-CC) or utilizing pigments prepared wherein the wash step with MEK prior to conversion was omitted. Thus, the devices tested were as follows: Device 9 was a drum having a 11 μm CTL and a CGL prepared with a Type V HOGaPc pigment obtained from a Type I HOGaPc that had been washed in accordance with the present disclosure with methyl ethyl ketone (MEK) prior to its conversion to Type V HOGaPc; Device 10 was a drum having a 13 μm CTL and a CGL prepared with a Type V HOGaPc pigment obtained from a Type I HOGaPc that had been washed in accordance with the present disclosure with MEK prior to its conversion to Type V HOGaPc; Device 11 was a drum having a 11 μm CTL and a CGL utilizing HOGaPc-RC pigment; Device 12 was a drum having a 13 μm CTL and a CGL utilizing HOGaPc-RC pigment; Device 13 was a drum having a 11 μm CTL and a CGL prepared with a Type V HOGaPc pigment obtained from a Type I HOGaPc that had not been washed in accordance with the present disclosure with MEK prior to its conversion to Type V HOGaPc; Device 14 was a drum having a 13 μm CTL and a CGL prepared with a Type V HOGaPc pigment obtained from a Type I HOGaPc that had not been washed in accordance with the present disclosure with MEK prior to its conversion to Type V HOGaPc; Device 15 was a drum having a 11 μm CTL and a CGL utilizing HOGaPc-CC pigment; and Device 16 was a drum having a 13 μm CTL and a CGL utilizing HOGaPc-CC pigment.

Electrical properties of these devices were determined as described above in Example 1 and CDS performance was evaluated and graded as described above in Example 1: dv/dx and CDS are as described in Example 1; Vdep is the voltage depletion of a device; Ver is the voltage erase of a device, i.e., the surface potential of a device after it has been subjected to an erase step of exposure wavelength of 680 nm at an intensity of about 50 to about 200 ergs/cm², and DTHK is the dielectric thickness obtained from the slope of plot of surface charge density versus surface potential of imaging member.

TABLE 2
Electrical Properties and CDS Performance
of the experimental devices

| Device | dV/dX | Ver | DTHK | Vdep | CDS |
|---|---|---|---|---|---|
| Device 9 | 191 | 18 | 4.0 | 35 | 3 |
| Device 10 | 225 | 11 | 4.9 | 47 | 2 |
| Device 11 | 190 | 18 | 4.1 | 30 | 5 |
| Device 12 | 225 | 11 | 5.0 | 35 | 4 |
| Device 13 | 194 | 19 | 4.1 | 31 | 5 |
| Device 14 | 226 | 13 | 4.9 | 36 | 4 |
| Device 15 | 201 | 18 | 4.1 | 34 | 4 |
| Device 16 | 238 | 12 | 5.0 | 44 | 3 |

All of the devices showed nominal behaviors (see Table 1). As can be seen from the above data, the pigment subjected to washing with MEK after hydrolysis but prior to conversion had the best grade (lowest) of the devices, suggesting the additional washing step produced a more amorphous pigment. Where the charge transport layer had a thickness of about 11 μm, the MEK washed pigment had a background grade of about 3, a significant 1 level lower than the device with the control pigment of HOGaPC-CC. For either Type I HOGaPc-RC or lab produced Type I without the MEK wash, the CDS performance was not as good as the HOGaPc-CC pigment.

The above methods were utilized to improve CDS. The CDS grade obtained for the resulting toner was one grade lower that HOGaPc-CC pigment at a very severe testing condition, i.e., high electric field (11 μm thick charge transporting layer), low processing speed, and in a high humidity and temperature zone.

### Example 3

The following experiment was carried out wherein Type I HOGaPc was converted into high sensitivity Type V HOGaPc. About 3 grams of Type I HOGaPc were placed into a 125 ml amber bottle with about 30 grams DMF and about 120 grams of 1 mm diameter HiBea borosilicate glass beads. Additional samples were prepared where a fraction of the DMF was replaced with MEK with concentrations of DMF to MEK of about 75/25, about 66/34, about 50/50, and about 33/67. The samples were roll milled at about 60 revolutions per minute for about 120 hours. The samples were then collected with suction filtration through a fritted glass filter having pores from about 4 μm to about 8 μm and rinsed with Acetone. The pigment cake was then dried under about 30 Torr vacuum at about 90° C. for about 24 hours. The final dry product was Type V HOGaPc.

About 2.5 grams of the Type V HOGaPc produced above for each of the varying concentrations of DMF and MEK, that is, about 100% DMF, and DMF/MEK at concentrations of about 75/25, about 66/34, about 50/50, and about 33/67, were mixed with about 32.5 grams of a combination of carboxyl-modified vinyl chloride/vinyl acetate copolymer (VMCH, commercially available from Dow Chemical) and n-butyl acetate (NBA) (VMCH/NBA) at about 5% so that the pigment:binder ratio was about 60:40 and about 12% solids content. The resulting combination of pigment and binder was charged into a lab size attritor with about 130 grams of about 1 mm diameter glass beads. The dispersions were monitored for particle size reduction via relative scattering index (RSI). RSI is about 100 times the ratio between the absorbance at about 830 nm to the absorbance at about 1000 nm. Absorbance was obtained utilizing a U-2000 UV-spectrometer by Hitachi. The particle size reduction of the resulting charge generation layer dispersions was deemed finished when the RSI value was below about 10. The dispersions were then diluted to about 5% solids content with NBA and filtered through about 20 μm filter cloth.

The resultant charge generation layer dispersions were Tsukiage coated on a 30 mm diameter aluminum drum. The drum was first coated with about 1.1 μm of an undercoat layer (UCL) including a polyvinyl butyral in combination with an organo zirconium compound prior to the application of the dispersion to produce a charge generation layer (CGL). The CGL dispersion was then applied at a rate of about 200 mm/min. An arylamine charge transfer layer (CTL) was dip coated at a thickness of about 13 μm. The drums were submitted for electrical scanning and print testing using the scanner described in Example 1 above. In addition to the devices having the charge generation layers formed with varying concentrations of DMF and MEK utilized in converting the Type I HOGaPc to the Type V HOGaPc, a control device was prepared using HOGaPc-RC as described above in Example 1 in the charge generation layer.

The FIGURE summarizes the results from both the electrical scanning and the print testing completed on the photoreceptors. An improvement was observed in background grade in every sample (those prepared with 100% DMF, and DMF/MEK at concentrations of about 75/25, about 66/34, about 50/50, and about 33/67) when compared to the HOGaPc-RC control. The electrical sensitivity of the pigment was also improved. In particular, the sample made with about 75% DMF and about 25% MEK provided the lowest background level while still maintaining a sensitivity that matched that of the 100% DMF sample.

It will be appreciated that various of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

What is claimed is:

1. A process comprising:
contacting a gallium phthalocyanine in an acid solution with a basic aqueous media to form a slurry;
contacting the slurry with a washing agent selected from the group consisting of ketones, ethers, alkanes, glycols, alcohols, aromatics, and pyridines to form a pigment; and
contacting the resulting pigment with a solvent system comprising at least two solvents of polar aprotic solvents, esters, and ketones.

2. A process in accordance with claim 1, wherein the washing agent is selected from the group consisting of alkyl alkyl ketones, dialkyl ethers, alkanes having from about 10 to about 40 carbon atoms, alkylene glycols, alcohols having from about 2 to about 20 carbon atoms, substituted aromatics having from about 1 to about 4 functional groups, alkyl pyridines, and combinations thereof.

3. A process in accordance with claim 1 wherein said gallium phthalocyanine is selected from the group consisting of halogallium phthalocyanines and alkoxy-bridged gallium phthalocyanines, the washing agent is selected from the group consisting of ethyelene glycols, etc; carbon chain lengths methyl ethyl ketone, diethyl ether, dodecane, ethylene glycol, propylene glycol, ethanol, propanol, toluene, xylene, 2-methylpyridine, 2-ethyl pyridine, and combinations thereof, and the at least two solvents comprise from about 2 to about 7 solvents.

4. A process in accordance with claim 1 wherein the acid solution is at a molar concentration from about 5 molar to about 30 molar and is selected from the group consisting of hydrogen halides, oxyacids of halogens and organic sulfonic acids, and the basic aqueous media comprises an aqueous hydroxide at a molar concentration of from about 3 molar to about 15 molar.

5. A process in accordance with claim 1 wherein the acid solution is selected from the group consisting of sulfuric acid, hydrochloric acid, hydrobromic acid, hydroiodic acid, chloric acid, perchloric acid, bromic acid, perbromic acid, iodic acid, periodic acid, nitric acid, trifluoroacetic acid, methanesulfonic acid, ethanesulfonic acid, propanesulfonic acid, pentanesulfonic acid, hexanesulfonic acid, heptanesulfonic acid, pyridinesulfonic acid, chloroethanesulfonic acid, bromoethanesulfonic acid, 1-diazo-2-naphthol-4-sulfonic acid, 3-hydroxypropane-1-sulfonic acid, aniline sulfonic acid, and combinations thereof at a molar concentration of from about 10 molar to about 20 molar, and the basic aqueous media is selected from the group consisting of ammonia and aqueous sodium hydroxide at a molar concentration of from about 6 molar to about 10 molar.

6. A process in accordance with claim 1 wherein the polar aprotic solvent is selected from the group consisting of N,N-dimethylformamide, N-methylpyrrolidone, dimethyl sulfoxide, acetonitrile, and mixtures thereof, the ester is selected from the group consisting of n-butyl acetate, ethyl acetate, and mixtures thereof, and the ketone is selected from the group consisting of acetone, methyl ethyl ketone, methyl isobutyl ketone, and mixtures thereof.

7. A process in accordance with claim 1 wherein the at least two solvents comprise from about 1 percent by weight to about 99 percent by weight of a polar aprotic solvent, optionally in combination with from about 99 percent by weight to about 1 percent by weight of an ester, optionally in combination with from about 5 percent by weight to about 80 percent by weight of a ketone.

8. A process in accordance with claim 1 wherein the at least two solvents comprise from about 20 percent by weight to about 80 percent by weight of a polar aprotic solvent, optionally in combination with from about 20 percent by weight to about 50 percent by weight of an ester, optionally in combination with from about 10 percent by weight to about 67 percent by weight of a ketone.

9. A process in accordance with claim 1 wherein the at least two solvents comprise N,N-dimethylformamide in combination with methyl ethyl ketone.

10. A process in accordance with claim 1 wherein a Type V hydroxygallium phthalocyanine is formed having particles with a surface area of from about 5 m²/g to about 120 m²/g and major peaks at Bragg angles (2 theta±0.2°) of 7.2, 10, 16.8, 18.6, 24, 25.3, 26.8, 28.3, 32.5 and with the highest peak at 7.2 degrees.

11. A process in accordance with claim 1 further comprising milling the pigment and solvent system for a period of time from about 2 hours to about 2 weeks, at a rolling speed from about 30 rpm to about 150 rpm, at a temperature from about 0° C. to about 40° C. wherein a Type V hydroxygallium phthalocyanine is formed having particles with a surface area of from about 30 m²/g to about 80 m²/g.

12. A process in accordance with claim 1 further comprising milling the pigment and solvent system for a period of time from about 72 hours to about 1 week, at a rolling speed from about 50 rpm to about 70 rpm, at a temperature from about 10° C. to about 30° C.

13. A process in accordance with claim 1 further comprising subjecting the pigment and solvent system to ultrasound of from about 0.5 MHz to about 10 MHz.

14. A process comprising:
contacting an alkoxy-bridged gallium phthalocyanine in an acid solution selected from the group consisting of hydrogen halides, oxyacids of halogens and organic sulfonic acids, with ammonia to form a pigment slurry;
concentrating the pigment slurry by filtration to obtain a pigment filtrate;
contacting the pigment filtrate with a washing agent selected from the group consisting of ketones, ethers, alkanes, glycols, alcohols, aromatics, and pyridines to obtain a pigment; and
contacting the resulting pigment with a solvent system comprising at least two solvents selected from at least two of the groups consisting of a polar aprotic solvent selected from the group consisting of N,N-dimethylformamide, N-methylpyrrolidone, dimethyl sulfoxide, acetonitrile, and mixtures thereof, an ester selected from the group consisting of n-butyl acetate, ethyl acetate, and mixtures thereof, and a ketone selected from the group consisting of acetone, methyl ethyl ketone, methyl isobutyl ketone, and mixtures thereof.

15. A process in accordance with claim 14 wherein the acid solution is selected from the group consisting of sulfuric acid, hydrochloric acid, hydrobromic acid, hydroiodic acid, chloric acid, perchloric acid, bromic acid, perbromic acid, iodic acid, periodic acid, nitric acid, trifluoroacetic acid, methanesulfonic acid, ethanesulfonic acid, propanesulfonic acid, pentanesulfonic acid, hexanesulfonic acid, heptanesulfonic acid, pyridinesulfonic acid, chloroethanesulfonic acid, bromoethanesulfonic acid, 1-diazo-2-naphthol-4-sulfonic acid, 3-hydroxypropane-1-sulfonic acid, aniline sulfonic acid, and combinations thereof at a molar concentration of from about 5 molar to about 30 molar, the ammonia is at a molar concentration of from about 3 molar to about 15 molar, the at least two solvents comprise from about 2 to about 7 solvents, the polar aprotic solvent comprises N,N-dimethylformamide, the ester comprises n-butyl acetate, and the ketone comprises methyl ethyl ketone.

16. A process in accordance with claim 14 wherein the washing agent comprises methyl ethyl ketone and a Type V hydroxygallium phthalocyanine is formed having particles with a surface area of from about 5 m²/g to about 120 m²/g and major peaks at Bragg angles (2 theta±0.2°) of 7.2, 10, 16.8, 18.6, 24, 25.3, 26.8, 28.3, 32.5 and with the highest peak at 7.2 degrees.

17. A process in accordance with claim 14 further comprising milling the pigment slurry and solvent system for a period of time from about 2 hours to about 2 weeks, at a rolling speed from about 30 rpm to about 150 rpm, at a temperature from about 0° C. to about 40° C., optionally further comprising subjecting the pigment slurry and solvent system to ultrasound of from about 0.5 MHz to about 10 MHz, wherein a Type V hydroxygallium phthalocyanine is formed having particles with a surface area of from about 30 m²/g to about 80 m²/g.

18. A photoreceptor comprising a photogenerating layer comprising a resin and a photogenerating component comprising a hydroxygallium phthalocyanine prepared by contacting a gallium phthalocyanine in an acid solution with a basic aqueous media to form a pigment slurry, contacting the pigment slurry with a washing agent selected from the group consisting of ketones, ethers, alkanes, glycols, alcohols, aromatics, and pyridines to form a pigment, and contacting the resulting pigment with at least two solvents selected from at least two of the groups consisting of polar aprotic solvents, esters, and ketones.

19. The photoreceptor of claim 18 wherein the gallium phthalocyanine is selected from the group consisting of halogallium phthalocyanines and alkoxy-bridged gallium phthalocyanines, the acid solution is selected from the group consisting of sulfuric acid, hydrochloric acid, hydrobromic acid, hydroiodic acid, chloric acid, perchloric acid, bromic acid, perbromic acid, iodic acid, periodic acid, nitric acid, trifluoroacetic acid, methanesulfonic acid, ethanesulfonic acid, propanesulfonic acid, pentanesulfonic acid, hexanesulfonic acid, heptanesulfonic acid, pyridinesulfonic acid, chloroethanesulfonic acid, bromoethanesulfonic acid, 1-diazo-2-naphthol-4-sulfonic acid, 3-hydroxypropane-1-sulfonic acid, aniline sulfonic acid, and combinations thereof at a molar concentration of from about 5 molar to about 30 molar, the basic aqueous media is selected from the group consisting of ammonia and aqueous sodium hydroxide at a molar concentration of from about 3 molar to about 15 molar, the at least two solvents comprise from about 2 to about 7 solvents, the polar aprotic solvent is selected from the group consisting of N,N-dimethylformamide, N-methylpyrrolidone, dimethyl sulfoxide, acetonitrile, and mixtures thereof, the ester is selected from the group consisting of n-butyl acetate, ethyl acetate, and mixtures thereof, and the ketone is selected from the group consisting of acetone, methyl ethyl ketone, methyl isobutyl ketone, and mixtures thereof, wherein a Type V hydroxygallium phthalocyanine is formed having particles with a surface area of from about 5 m²/g to about 120 m²/g and major peaks at Bragg angles (2 theta±0.2°) of 7.2, 10, 16.8, 18.6, 24, 25.3, 26.8, 28.3, 32.5 and with the highest peak at 7.2 degrees.

20. The photoreceptor of claim 18, wherein the washing agent is selected from the group consisting of methyl ethyl ketone, diethyl ether, dodecane, ethylene glycol, propylene glycol, ethanol, propanol, toluene, xylene, 2-methylpyridine, 2-ethyl pyridine, and combinations thereof, and further comprising a charge transport layer, an optional substrate, an optional hole blocking layer, and an optional adhesive layer, and wherein the thickness of the photogenerating layer is from about 0.05 microns to about 10 microns, the thickness of the charge transport layer is from about 2 micrometers to about 50 micrometers, and the charge transport layer comprises hole transport molecules comprising an aryl amine.

21. The photoreceptor of claim 20, wherein the charge transport layer comprises hole transport molecules comprising an aryl amine of the formula 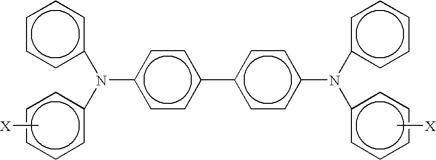
wherein X is selected from the group consisting of alkyl, halogen, alkoxy or mixtures thereof.

22. A process comprising:
contacting a gallium phthalocyanine with an acid solution at a molar concentration of from about 5 molar to about 30 molar and a basic aqueous media having a molar concentration from about 3 molar to about 15 molar to form a slurry;
contacting the slurry with a suitable washing agent; and
contacting the resulting pigment with a solvent system comprising at least two polar aprotic solvents, esters, and ketones.

23. A hydroxygallium phthalocyanine obtained by the process of claim 1.

24. The hydroxygallium phthalocyanine of claim 23, wherein the hydroxygallium phthalocyanine has a particle size from about 50 nm to about 150 nm.
